(19) United States
(12) Patent Application Publication
(10) Pub. No.: US 2023/0136100 A1
(43) Pub. Date: May 4, 2023

(54) ENDOSCOPE WITH AUTOMATIC STEERING

(71) Applicant: Covidien LP, Mansfield, MA (US)

(72) Inventors: Derek Scot TATA, Boulder, CO (US); Peter Douglas Colin INGLIS, Boulder, CO (US)

(73) Assignee: Covidien LP, Mansfield, MA (US)

(21) Appl. No.: 18/050,013

(22) Filed: Oct. 26, 2022

Related U.S. Application Data
(60) Provisional application No. 63/274,262, filed on Nov. 1, 2021.

(51) Int. Cl. A61B 1/00; A61B 1/05

(52) U.S. Cl. CPC A61B 1/00009; A61B 1/05

(57) ABSTRACT

An endoscope automatic steering system is provided. An endoscope controller receives an image signal from an endoscope and identifies a passage based on the image signal. A steering controller selects a steering target within the passage and generates steering instructions to cause the endoscope to automatically steer towards the steering target.

### CROSS-REFERENCE TO RELATED APPLICATIONS

This application claims the benefit of U.S. Provisional Application No. 63/274,262 filed Nov. 1, 2021, entitled “Endoscope with Automatic Steering,” which is incorporated herein by reference in its entirety.

### BACKGROUND

The present disclosure relates generally to medical devices and, more particularly, to endoscope navigation and steering techniques that use automatic steering based on tracking of a navigation target through analysis of live endoscope images and related methods and systems.

Medical endoscopes are long, flexible instruments that can be introduced into a cavity of a patient during a medical procedure in a variety of situations to facilitate visualization and/or medical procedures within the cavity. For example, one type of scope is an endoscope with a camera at its distal end. The endoscope can be inserted into a patient’s mouth, throat, trachea, esophagus, or other cavity to help visualize anatomical structures, or to facilitate procedures such as intubations, biopsies, or ablations. The endoscope may include a steerable distal end that can be actively controlled to bend or turn the distal end in a desired direction, to obtain a desired view or to navigate through anatomy. Navigating the endoscope into a patient’s airway and through a curved path past the vocal cords into the trachea can be challenging.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In an embodiment, an endoscope automatic steering system is provided. The system includes an endoscope comprising a distal end with a camera producing an image signal and an endoscope controller coupled to the endoscope. The endoscope controller receives the image signal from the endoscope; identifies, via a feature identification model, an one anatomical feature in the image signal; selects a steering target based on the identified anatomical feature; and automatically steers the distal end towards the steering target during distal motion of the distal end of the endoscope.

In an embodiment, an endoscope automatic steering system is provided. The system includes an endoscope comprising a steerable distal end with a camera producing an image signal and an orientation sensor producing an orientation signal of an orientation of the steerable distal end and an endoscope controller. The endoscope controller receives the image signal and the orientation signal; automatically selects a steering target of the endoscope based on the image signal; identifies a distal advancement of the endoscope based on the orientation signal or the image signal or both; and generates instructions to automatically steer the distal end of the endoscope towards the steering target during the distal advancement.

In an embodiment, an endoscope automatic steering method is provided that includes the steps of automatically steering an endoscope towards a steering target in a passage of a subject; receiving a user steering input to actively steer the endoscope; pausing automatically steering of the endoscope based on the user steering input; and resuming automatically steering the endoscope towards the steering target after a predetermined time period has passed or no additional user steering inputs are received

Features in one aspect or embodiment may be applied as features in any other aspect or embodiment, in any appropriate combination. For example, features of a system, handle, controller, processor, scope, method, or component may be implemented in one or more other system, handle, controller, processor, scope, method, or component.

### BRIEF DESCRIPTION OF THE DRAWINGS

Advantages of the disclosed techniques may become apparent upon reading the following detailed description and upon reference to the drawings in which:

FIG. 1 is a view of an endoscope system, according to an embodiment of the disclosure.

FIG. 2 is a block diagram of the endoscope system of FIG. 1, according to an embodiment of the disclosure;

FIG. 3 is a flow diagram of an automatic steering method, according to an embodiment of the disclosure;

FIG. 4 is a schematic illustration of automatic steering of an endoscope that tracks alignment to a center of a passage, according to an embodiment of the disclosure;

FIG. 5 is a flow diagram of an automatic steering method using image segmentation, according to an embodiment of the disclosure;

FIG. 6 is an example segmented image, according to an embodiment of the disclosure;

FIG. 7 is a flow diagram of an automatic steering method using object detection, according to an embodiment of the disclosure;

FIG. 8 is an example image with a detected object, according to an embodiment of the disclosure;

FIG. 9 is an example image with multiple detected objects, according to an embodiment of the disclosure;

FIG. 10 is a flow diagram of a candidate selection steering method, according to an embodiment of the disclosure;

FIG. 11 is a flow diagram of an automatic steering method in conjunction with endoscope movement, according to an embodiment of the disclosure;

FIG. 12 is a flow diagram of an automatic steering method with a user override, according to an embodiment of the disclosure;

FIG. 13 is a schematic illustration of a controller display screen with user steering input icons and in which the user is providing no user steering input via the icons, according to an embodiment of the disclosure;

FIG. 14 is a schematic illustration of a controller display screen with user steering input icons and in which the user provides user steering input via the icons to override automatic steering, according to an embodiment of the disclosure;

FIG. 15 is a flow diagram of an automatic steering method with a detected steering override, according to an embodiment of the disclosure;

FIG. 16 is a flow diagram of an automatic steering method that automatically activates when in a subject, according to an embodiment of the disclosure; and

FIG. 17 is a block diagram of an endoscope system, according to an embodiment of the disclosure.

### DETAILED DESCRIPTION OF SPECIFIC EMBODIMENTS

A medical scope or endoscope as provided herein is a thin, elongated, flexible instrument that can be inserted into a body cavity for exploration, imaging, biopsy, or other clinical treatments, including catheters, narrow tubular instruments, or other types of scopes or probes. Endoscopes may be navigated into the body cavity (such as a patient’s airway, gastrointestinal tract, oral or nasal cavity, or other cavities or openings) via advancement of the distal end to a desired position and, in certain embodiments, via active steering of the distal end of the endoscope. Endoscopes may be tubular in shape.

Advancement of long, flexible medical devices into patient cavities is typically via force transferred from a proximal portion of the device (outside of the patient cavity), that results in advancement of the distal end within the patient cavity. As used herein, “proximal” refers to the direction out of the patient cavity, back toward the handle end of a device, and “distal” refers to the direction forward into the patient cavity, away from the doctor or caregiver, toward the probe or tip end of the device. For example, a doctor or other operator holding a proximal portion of the endoscope outside of the patient cavity pushes downward or forward, and the resulting motion is transferred to the distal end of the endoscope, causing the tip to move forward (distally) within the cavity. Similarly, a pulling force applied by the operator at the proximal portion may result in retreat of the distal end or movement in an opposing (proximal) direction out of the patient cavity. In addition, the operator can change the orientation of the distal end of the endoscope by twisting or angling of the proximal portion the endoscope to cause a corresponding change in the orientation of the distal end.

In some cases, an endoscope can include steering capabilities, such that the operator can input steering commands into a handheld control device, and the steering commands are translated into actuation of the distal end of the endoscope in the desired direction. Thus, using the steering commands, the operator can more precisely orient the distal end of the endoscope in the desired direction. However, while operator-controlled steering may provide more precise positioning of the distal end, the overall speed of endoscope navigation to a desired target can be dependent of the operator’s skill at using the steering commands and the operator’s ability to manually steer an efficient pathway. In one example, an endoscope is used to directly visualize the airway during an intubation to aid in passing an endotracheal tube into the trachea.

Visualization using the endoscope camera provides a direct view of the airway, which can be used in addition to or instead of laryngoscope imaging, which provides images from a laryngoscope camera inserted into the upper airway and manipulated by the operator. Use of a laryngoscope can result in partial opening or straightening of airway passages due to patient positioning and force applied to the laryngoscope to lift the patient’s jaw. However, if the operator is not using a laryngoscope to open or lift the patient’s jaw, the patient’s upper airway passages may be more curved or closed. Thus, endoscope-based navigation may involve steering challenges through more curved airway passages. For example, oversteering through a curved passage of the vocal cords can result in the endoscope passing the vocal cords at an off-angle within the tracheal passage, thus requiring additional course corrections that add time to the endoscopy procedure.

Provided herein are automatic or assisted steering techniques that track a steering target. In an embodiment, the automatic steering techniques use artificial intelligence or machine learning algorithms applied to live endoscope images to identify features within the endoscope images and automatically select an identified feature as a steering target. For example, the automatic steering techniques can identify an airway passage within an endoscope image or images and automatically steer the distal end of the endoscope to maintain an orientation towards a center of the airway passage. Further, in embodiments, the automatic steering operates in real-time along with the progress of the endoscope, automatically adjusting the selected navigation target (e.g., the center of the passage) as new endoscope images are received. In an embodiment, the endoscope automatically steers the distal end toward a target during forward motion, without the user having to manually steer (bend) the distal end.

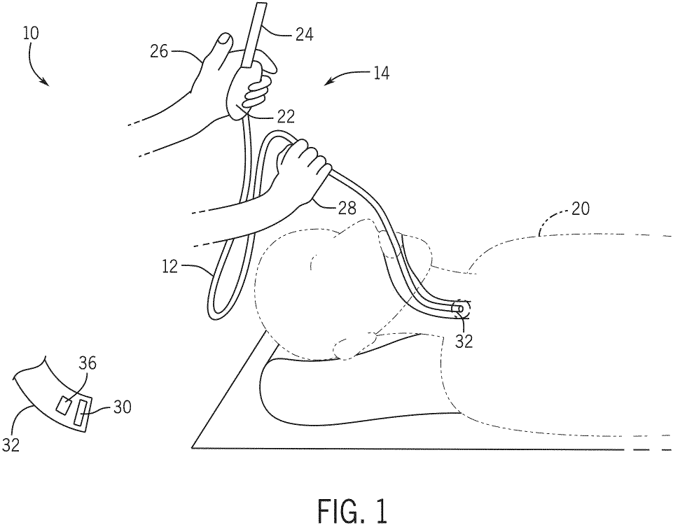

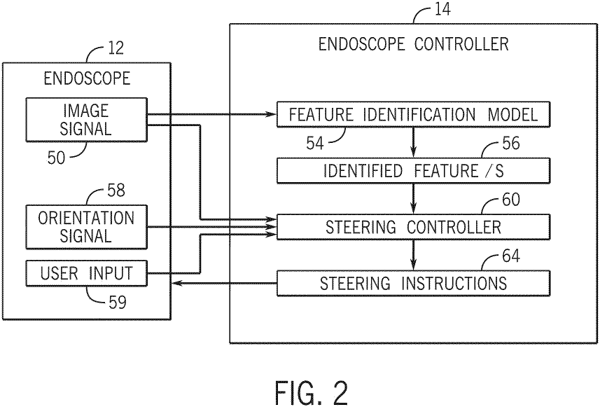

An example automatic steering system is depicted in FIG. 1. In the embodiment shown, the endoscope viewing system 10 includes an endoscope 12 connected to an endoscope controller 14. The endoscope controller 14 can, in embodiments, use a steering controller (see FIG. 2) as discussed in the disclosed embodiments to generate steering instructions for the endoscope 12 as generally discussed herein.

The endoscope 12 is being inserted into a patient 20 during a clinical procedure. As shown in FIG. 1, the endoscope 12 is an elongated, tubular scope that is connected at its proximal end to the controller 14. The controller 14 includes a handle, puck, or wand 22 with a display screen 24. The display screen shows images from a camera 30 at the distal end 32 of the endoscope 12, within the patient cavity. The clinician (the operator) who is operating the endoscope 12, holds the handle 22 with his or her left hand 26, and grips or pinches the endoscope 12 with his or her right hand 28. The operator can move the endoscope 12 proximally or distally with the right hand, while watching the resulting images from the camera on the display screen 24. In an embodiment, the display screen 24 is a touch screen, and the operator can input touch inputs on the screen 24 (such as with the operator’s left thumb) to steer the distal end of the endoscope 12, such as to bend it right, left, up, or down.

While embodiments of the disclosure are discussed in the context of activation of automatic steering based on acquired airway images from the endoscope 12, it should be understood that the acquired airway images may or may not be concurrently displayed on the display screen 24 while the automatic steering is active. For example, the controller 14 may be implemented as a video laryngoscope that receives laryngoscope images. The display screen 24 can display one or both of the endoscope images or the laryngoscope images. However, even when the controller 14 is in a laryngoscope image display mode and no endoscope images are displayed, the endoscope images from the endoscope 12 can be used to generate steering instructions. However, in an embodiment, switching from the laryngoscope display to the endoscope full screen display can act as a trigger to initiate automatic steering.

Additionally or alternatively, the endoscope 12 can be steered automatically based on instructions from the controller 14 provided by a steering controller 60. As shown in the illustrated block diagram of FIG. 2 and with reference to FIG. 1, the steering controller 60 can be implemented on the controller 14. That is, operations of the steering controller 60 as well as feature identification may be executed by the controller 14 on the handle 22. The controller 14 receives an image signal 50 as an input. The endoscope controller 14 may include a feature identification model 54 that uses the image signal 50 to output one or more identified features 56 in the image signal 50. The feature identification model 54 may incorporate artificial intelligence or machine learning algorithms to identify one or more features 56 as generally discussed herein.

The identified features can be provided, in an embodiment, to a steering controller 60. In an embodiment, the steering controller 60 uses a rules-based algorithm and parameters of the endoscope actuators to generate steering instructions 64 to steer towards at least one identified feature 56 or keep the at least one identified feature 56 in a center of the image according to the rules of the steering controller 60. Accordingly, based at least on the image signal 50, the steering controller 60 generates steering instructions 64 that are provided from the endoscope controller 14 to the endoscope 12 to cause the distal end 32 of the endoscope 12 to be automatically steered according to the steering instructions 64.

In an embodiment, the feature identification model 54 analyzes the image signal 50 to identify one or more anatomical features in the images in the image signal 50. In an embodiment, the anatomical features are a patient passage (e.g., an airway passage), a center of a passage, passage walls, particular anatomical structures (e.g., teeth, tongue, upper airway, vocal cords, carina, polyps, lesions, and other internal structures), specific portions (such as a side or center) of these features, and/or combinations of these features. In an embodiment, the feature identification model 54 may differentiate between negative spaces (vocal cords, trachea, esophagus, etc.) and including positive features (epiglottis, arytenoids, etc.). In one example, the feature identification model 54 identifies a passage (e.g., a lumen) or non-lumen feature. The feature identification model 54 may identify multiple candidate features and select a candidate.

The steering controller 60 generates steering instructions 64 to steer the distal end 32 relative to the identified features 56. For example, in an embodiment, the steering controller 60 steers the distal end 32 away from passage walls, towards a center of a passage, and/or towards an identified anatomical structure, such as a carina or vocal cords. The steering instructions 64 cause the distal end 32 to be steered within the subject 20 without additional input from the operator.

The image signal 50 is generated by the camera 30 of the endoscope 12. In embodiments, the image signal 50 is a raw image signal. In embodiments, the image signal 50 may undergo preprocessing. For example, the image signal 50 may be scaled or oriented to a reference frame of the operator of the controller 14. Thus, in embodiments, in addition to the image signal 50, the steering controller 60 receives an orientation signal 58 from the orientation sensor 36 as an input. Further, as discussed herein, the steering controller 60 may take endoscope axial or distal movement, such as distal movement during endoscope insertion, into account in generating the steering instructions 64, and the endoscope movement may be determined based on the orientation signal 58. The steering controller 60 may use the image signal 50, the identified features 56, and, in an embodiment, any received user steering inputs 59, as inputs to the algorithm generate steering instructions.

In an embodiment, the disclosed techniques use automatic steering with object or target tracking, rather than motion tracking. Motion tracking steers the distal end of the endoscope along a motion vector, to point the camera along a particular direction of motion of the endoscope. With motion tracking, steering is based on aiming the distal end in the direction of motion, such as through pixel flow or vanishing point analysis. Motion tracking can involve challenges with tracking motion between frames in the image signal to identify the desired direction of motion for steering. By contrast, target tracking points the distal end of the endoscope toward a target identified in the image signal. The target that can be identified in successive still image frames, without tracking a direction of motion between those image frames.

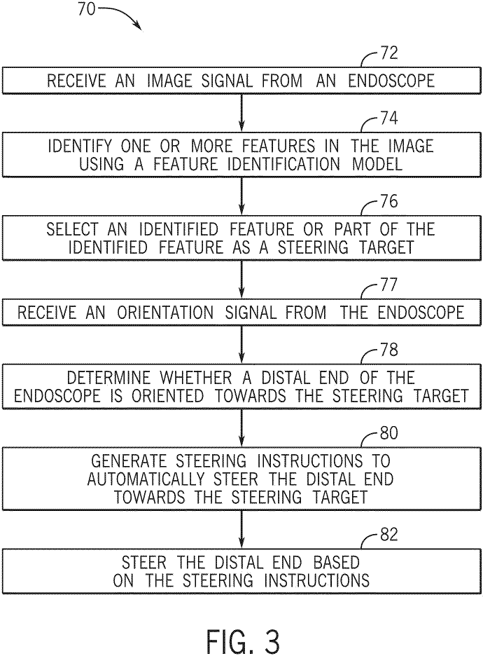

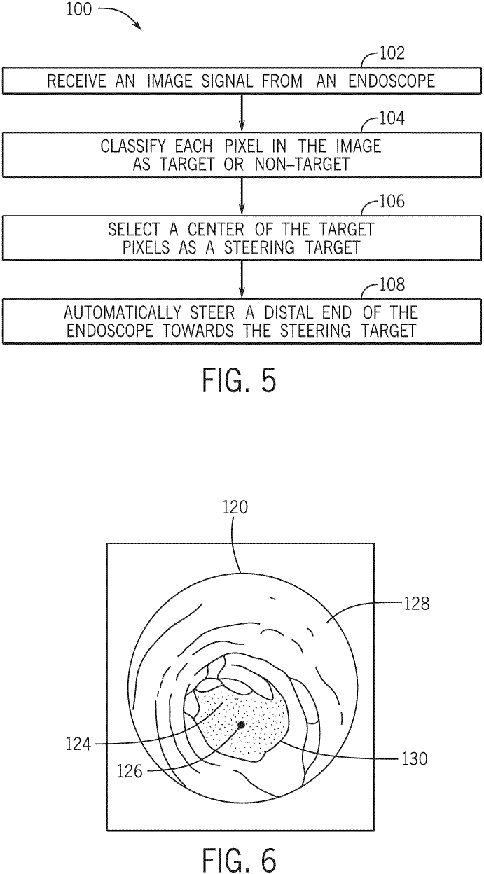

FIG. 3 is a flow diagram of a target tracking automatic steering method 70 that can be used in conjunction with the system 10 and with reference to features discussed in FIGS. 1-2, in accordance with an embodiment of the present disclosure. Certain steps of the method 70 may be performed by the endoscope controller 14. The method 70 initiates with receiving an image signal 50 from the endoscope 12 (block 72). The image signal 50 includes one or more images acquired by the camera 30 of the endoscope 12. The image signal 50 is provided to the steering controller 60, and, in an embodiment, the feature identification model 54 identifies a feature in the image (block 74), such as a passage (for example, a tracheal or bronchial passage) of the patient. For example, the feature identification model 54 can use object detection or image segmentation (discussed further below with reference to FIGS. 5-8) to identify the feature, such as the passage, using characteristics of the image signal. Once identified, the controller 14, e.g., using the steering controller 60, can select a portion (such as a center, or an approximate center) of the identified passage as a steering target for the endoscope 12 (block 76). In an embodiment, the passage has an irregularly shaped cross-section, and the approximate center of the passage is a centroid or center of cross-sectional area of the passage.

In certain cases, the image signal 50 includes multiple features that are identified by the feature identification model 54. For example, the branching of a pathway into at least two possible forward passages can be identified by the feature identification model 54 operating to identify any passages in the image signal 50. Thus, multiple features can be identified in the capture image of the image signal 50. Accordingly, block 76 may include selecting one feature as the steering target. In an embodiment, the feature selection is performed by the steering controller 60.

Once selected, the method 70 can receive an orientation signal 58 from the endoscope 12 (block 77). The orientation signal 58 may provide information about an orientation (e.g., a roll) of the endoscope 12 as well as information about movement of the endoscope 12 in a distal or proximal direction. The method 70 further determines whether the distal end 32 of the endoscope 12 is oriented towards the steering target. In an embodiment, the determination is based on the image signal 50. That is, the distal end 32 can be determined to be pointed away from steering target based on a position of the steering target in the image signal 50. When the steering target is not centered in the image of the image signal 50, the distal end 32 may be determined not to be oriented towards the steering target The steering controller 60 generates steering instructions 64 to automatically steer the distal end 32 towards the steering target (block 80). The steering instructions 64 are passed to the endoscope 12, and the distal end 32 is steered based on the steering instructions 64 (block 82).

When the distal end 32 of the endoscope 12 is determined to be oriented towards the steering target (block 84), the method 70 may generate steering instructions 64 (block 86) that maintain the orientation of the distal end 32, because the orientation of the distal end 32 does not require correction or adjustment. The method 70 can determine that the distal end 32 is oriented towards the steering target within a certain preset tolerance to avoid ping-ponging of the steering, which can cause the displayed image to have a jerky quality.

The automatic steering may, in embodiments, operate to track the steering target between individual frames in the image signal 50 to keep the selected steering target generally in the center of the endoscope image. In an embodiment, the steering controller 60 uses still images as inputs, and the steering targets can be linked between frames as part of target tracking. As new images are acquired by the camera 30, the method 70 iterates back to block 72. The steering controller can use a center or centroid tracking algorithm to correlate one or both of the identified passage or the steering target between frames. In target tracking, the system 10 can seek a steering target in the event that a target is not already in the memory. Further, the system 10 may iteratively purge or write over identified steering targets on a periodic basis as new or updated images signals 50 are received.

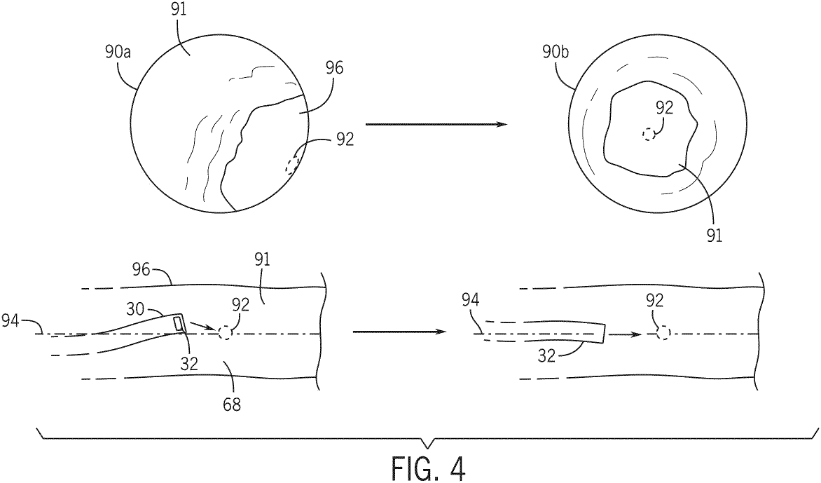

FIG. 4 shows a schematic illustration of automatic steering of the distal end 32 of the endoscope 12 based on steering instruction 64 from the steering controller 60. The top portion of FIG. 4 shows images 90 (i.e., images 90a, 90b) captured by the camera 30 during navigation within a passage 91 using automatic steering. The bottom portion shows a corresponding change in the orientation of the distal end 32 of the endoscope 12 within the passage 91 as a result of the automatic steering. Starting from the left side of FIG. 4, the distal end 32 is not oriented toward the target (the center 92 of the passage). Instead, in this example, the distal end is oriented towards the walls 96 of the passage 91 rather than being straight or generally oriented toward the center 92 (e.g., towards a point along a central axis 94). In this orientation, further distal movement could cause the endoscope to collide with the walls 96 of the passage, which could impede further distal movement, cause injury to the patient, and/or obscure the view from the camera 30. For example, this undesired orientation of the distal end 32 can be caused by the operator inadvertently oversteering, by the operator intentionally pausing distal movement and steering the camera to view the walls 96 or some other portion of the anatomy, or because of a natural curve of the passage 91. The corresponding image 90a is indicative of the resulting orientation, and the passage 91 is not centered within the image 90a.

When automatic steering is active to track a target feature (such as the center 92), the steering controller 60 can generate steering instructions that cause the distal end 32 to automatically bend, rotate, or move back toward the target. Using the image 60a as an input, the feature identification model 54 identifies the passage 91 (e.g., via identification of the walls 96 and/or identification of a negative space indicative of the passage 91 as generally discussed herein) and, in an embodiment, the endoscope controller, via the feature identification model 54 or the steering controller 60, estimates a location of a center 92 of the passage 91. The steering controller 60 generates steering instructions to point the distal end 32 towards the center 92. Execution of the steering instructions causes the distal end 32 to bend, move, or rotate toward the center 92 as shown by arrow A. After executing these instructions, the distal end 32 is generally oriented along the center axis 94 and pointed towards a location corresponding to the identified center 92. The corresponding image 90b is indicative of the distal end 32 being pointed towards the steering target, and the center 92 of the passage 91 is centered within the image 90b.

As provided herein, an endoscope controller 14 may include steering control that uses a feature identification model (e.g., feature identification model 54, FIG. 2). The feature identification model 54 may be a supervised or unsupervised model. In an embodiment, the feature identification model 54 may be built using a set of airway images and associated predefined passage and non-passage labels (which, in an embodiment, may be provided manually in a supervised machine learning approach). This training data, with the associated labels, can be used to train a machine classifier, so that it can later process the image signal 50.

Depending on the classification method used, the training set may either be cleaned, but otherwise raw data (unsupervised classification) or a set of features derived from cleaned, but otherwise raw data (supervised classification). In an embodiment, deep learning algorithms may be used for machine classification. Classification using deep learning algorithms may be referred to as unsupervised classification. With unsupervised classification, the statistical deep learning algorithms perform the classification task based on processing of the data directly, thereby eliminating the need for a feature generation step. The feature identification model may ues Haar cascades, Histogram of Gradients with Support Vector Machines (HOG + SVM), or a convolutional neural network. Features can be extracted from the set using a deep learning convolutional neural network, and the images can be classified using logistic regression, random forests, SVMs with polynomial kernels, XGBoost, or a shallow neural network. A best-performing model that most accurately correctly labels patient passages in the set of airway images is selected.

As discussed herein, the steering controller 60 can select a steering target based on identified features from an image signal 50 and generate instructions to steer towards the selected steering target. FIG. 5 is a flow diagram of an automatic steering method 100 using image segmentation that can be used in conjunction with the system 10 and with reference to features discussed in FIGS. 1-4, in accordance with an embodiment of the present disclosure. The method 100 initiates with receiving an image signal 50 including one or more images acquired by the camera 30 of the endoscope 12 (block 102). The image signal 50 is provided to the endoscope controller 14 for processing. The feature identification model 54 can classify each pixel in the image as target or non-target (block 104). For example, where the target is a passage (such as an airway passage), the classified “target” pixels are those that are likely to be within a passage, while the classified “non-target” pixels are those that are more likely to be passage walls. The method 100 selects a center of the target pixels as a steering target (block 106) and automatically steering the distal end of the endoscope towards the steering target (block 108). Each of these steps will be described in further detail below.

Regarding block 104, in one example, the feature identification model 54 can be trained on anatomy images of a population of subjects having categorized target and non-target pixels. Pixels that are likely to be target pixels of a passage may be relatively darker in color and part of a field of contiguous darker pixels that are at least partially bounded by lighter pixels that are likely to be non-target passage walls. Additional rules of the model 54 may include a range of likely passage sizes. For example, an airway passage in an image is likely to be at least a particular size in the image, which would exclude small darker shadings of the passage walls from being incorrectly categorized as target pixels.

FIG. 6 is an example a segmented image 120 showing categorized target pixels 124 highlighted and having a center 126. The controller applies a set of rules to the image to classify pixels in the image as target or non-target pixels. These rules may differ based on the type of anatomy being targeted. For example, in FIG. 6, the steering target is the center of a passage, and the highlighted pixels 124 have been identified as being within that target area. The non-target pixels are the non-highlighted pixels in the image 120, associated with the walls 128 or other structures. In an embodiment, the segmentation may be a semantic segmentation, e.g., Deeplab V3+, running at greater than 30 frames per second on our hardware. In an embodiment, the output of the steering controller segmentation is a matrix or mask image in which the target pixels have a different value than the non-target pixels. It should be noted that the target pixels (such as area 124 in FIG. 6) or targeted area (center 126) are not necessarily displayed to the user. Rather, the operation of the feature identification model 54 to characterize the pixels and identify a target can be done all or partially in the background, without view by an operator of the endoscope system 10. In that case, the pixel classification used to select the steering target, as well as the selection of the steering target, are steps that may not be visible to the operator. For example, marking the target pixels 124 and/or the center 126 on the image display 24 (shown in FIG. 1) may obscure anatomical details and interfere with the operator’s clinical care of the patient. However, in an embodiment, the center 126, e.g., the steering target, may be marked by an icon on the image display for navigation reference, as shown and discussed further below.

For a target area 124 having an irregular shape, the center 126 can be a centroid or approximate center. In an embodiment, the center 126 can be selected as a center of a circle having a best fit to a perimeter 130 of the target pixels 124. While the illustrated embodiment shows a steering target that is the center of a passage, other steering targets are also contemplated. For example, the steering target can be an edge of an identified feature or a portion of an identified feature. In one example, the identified feature can be patient vocal cords, and the steering target can be the space between the vocal cords.

The steering controller 60 can generate steering instructions 64 to cause the distal end 32 of the endoscope 12 to be oriented to the steering target. In one example, a current position and orientation of the distal end 32 relative to the steering target is determined. The distal end 32 can be, for example, oriented in a particular 360 direction that deviates from a desired orientation towards the steering target. The steering instructions cause rotation or bending of the distal end 32 to be pointed in the particular 360 direction that aligns with the steering target.

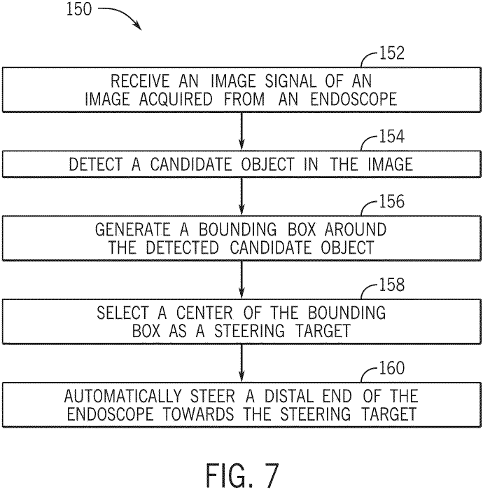

FIG. 7 is a flow diagram of another method 150 for automatically detecting a steering target within an image. The approach in FIG. 7 is based on object detection and can be used in conjunction with the system 10 and with reference to features discussed in FIGS. 1-4, in accordance with an embodiment of the present disclosure. The method 150 initiates with receiving an image signal 50 including one or more images acquired by the camera 30 of the endoscope 12 (block 152). The image signal 50 is provided to the steering controller 60 for processing. In contrast to segmentation-based techniques, in which each individual pixel is categorized, object detection techniques can use the steering controller 60 to analyze the image to detect candidate objects (block 154), e.g., a passage, and generate a bounding box around a detected object (block 156). In an embodiment, the bounding box is a smallest box that contains the detected object. The steering controller 60 selects a center of the bounding box as a steering target (block 158), and the system 10 automatically steers the distal end of the endoscope towards the steering target (block 160).

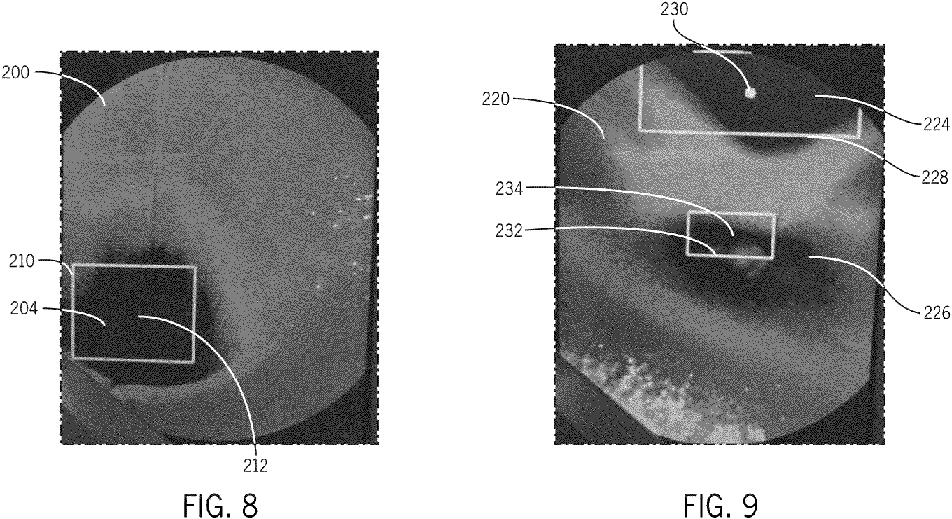

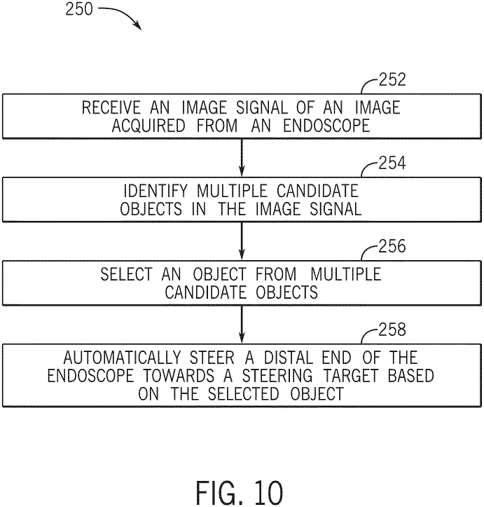

FIGS. 8-9 are example images showing detected objects. In FIG. 8, an airway image 200 is analyzed, and a first object 204 indicative of a passage is detected. The steering controller 60 generates a bounding box 210 around the detected object, and a center 212 of the bounding box is set as the steering target. FIG. 9 is an example image 220 showing a case with detection of multiple candidate objects, a first object 224 corresponding to a tracheal passage 224 and a second object 226 corresponding to an esophageal passage 226. Thus, each detected object prompts generation of a corresponding bounding box, shown here as first bounding box 228 with center 230, and second bounding box 232 with center 234. Where the steering controller 60 identifies multiple objects that are all candidates as potential steering targets, such as the respective centers 230, 234 of the bounding boxes 228, 232, the method may include ranking the candidate objects in order to select one as the steering target (see FIG. 10). For example, the automatic steering can distinguish between a tracheal passage and an esophageal passage based on additional characteristics of those anatomies, such as size. For example, tracheal passages tend to be larger than esophageal passages within a single patient. Here, the bounding box 228 of the tracheal passage 224 is larger than the bounding box 232 of the esophageal passage 226, and the steering controller 60 selects the center 230 of the larger bounding box as the steering target because it is more likely than the smaller box to be the tracheal passage. In another example, when multiple candidate objects (such as multiple passages) are detected, the system 10 automatically pauses steering and waits for user input to select one of the candidate objects as the steering target. One such user input is a tap (touch) input from the user on the screen on the desired steering target. Another such user input is a manual steering input in which the user manually steers the distal end 32 toward the desired target. After the input is detected (a touch input on the tracheal passage 224, or movement of the distal end 32 towards the tracheal passage 224 and away from the esophageal passage), the steering controller 60 sets the center 230 of the tracheal bounding box 228 as the steering target and reactivates automatic steering.

In an embodiment, the illustrated bounding boxes and selected centers are not visible on the images displayed to the operator on the display screen 24 (shown in FIG. 1), and the steering controller 60 generates the bounding boxes and selects respective centers without altering the displayed images. However, in an embodiment, one or both of the generated bounding box or the center of the bounding box is overlaid or otherwise marked on the displayed image on the screen 24, to inform the user which objects the steering system is considering as candidate objects and selecting as the steering target. The bounding boxes (or other visual indication of a candidate object) may also be displayed to the user in the case where the system identifies multiple candidate objects and pauses for user input, as discussed above.

As discussed herein, the feature identification model 54 may use segmentation, object identification, or other techniques to identify multiple candidate objects in the image signal 50. FIG. 10 is a flow diagram of a steering method 250 that can be used to select a best candidate object in conjunction with the system 10 and with reference to features discussed in FIGS. 1-4, in accordance with an embodiment of the present disclosure. In embodiments, certain steps of the method are performed by the endoscope controller 14, e.g., by one or more of the feature identification model 54 or the steering controller 60. The method 250 initiates with receiving an image signal 50 including one or more images acquired by the camera 30 (block 252). Using the feature identification model 54, the method 250 identifies two or more candidate objects in the image signal 50 (block 254). When multiple candidate objects are identified, the method can select a candidate object (block 256) from the multiple objects, e.g., a best or highest ranked object, and automatically steer a distal end of the endoscope towards a steering target based on the selected object (block 258).

The candidate may be selected based on a quality metric or ranking of the candidate objects. In one example, the last or most-recent set of identified features, including the last or most-recent steering target or selected candidate object, is provided to the method 250. The candidate object can be selected based on a highest likelihood of tracking to the most-recent steering target or most-recent selected candidate object. For example, each candidate object can be provided with an identification tag or number from the feature identification model 54. The identification tag of the candidate object in the image signal 50 that aligns with or is closest to the most-recent steering target is selected as the best candidate object, and the identification tag of the selected object can be provided to the memory to be used in tracking for subsequent image signals 50. If the orientation of the endoscope 12 has not changed significantly between frames, the previous or most-recent selected candidate object and the new selected candidate object may overlap or be positioned in a similar location within the image.

However, the orientation of the distal end 32 can change based on user input. For example, the user can swipe across the screen or otherwise interacts with user steering inputs to reorient the distal end 32. In such an example, the previously identified candidate object or objects may no longer be in the center of the image or in the image at all. Thus, the feature identification model 54 can identify new candidate objects and automatically select the candidate as discussed herein. In an embodiment, the steering controller 40 may present the candidate objects, e.g., the bounding boxes or indicators of potential steering targets, on the display screen 24, and the user can select the preferred steering target. Thus, the steering target can be selected based on user selection.

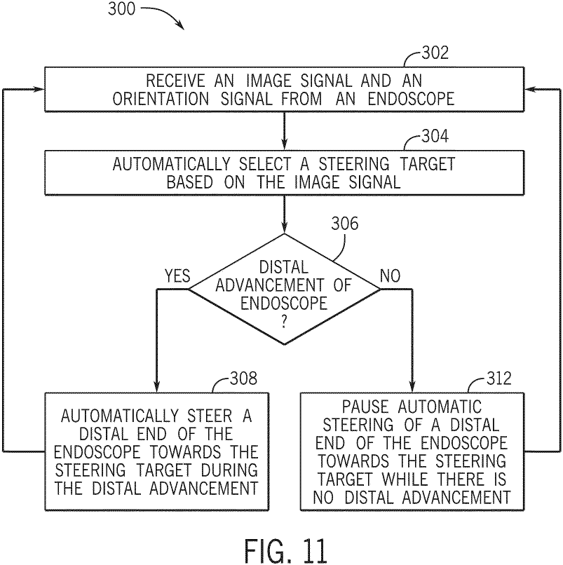

The automatic steering as disclosed herein may be part of an assisted steering system that permits varying degrees of automatic steering and user control of steering. In certain embodiments, the controller 14 has user-selectable options to select or deselect an automatic steering mode. In one example, user deselection of the automatic steering mode completely deactivates all automatic steering, and user selection of the automatic steering mode activates automatic steering and causes the controller 14 to use the steering controller 60 to automatically steer. However, even when automatic steering is activated (such as selected by the user or activated as a default), the system 10 may conditionally pause the automatic steering in a rules-based manner. FIGS. 11-16 are directed to embodiments of conditional activation, deactivation, or pausing of automatic steering.

In an embodiment, automatic steering is synchronized or coordinated with forward (distal) motion of the endoscope 12. FIG. 11 is a flow diagram of a motion-dependent automatic steering method 300 that can be used in conjunction with the system 10 and with reference to features discussed in FIGS. 1-4, in accordance with an embodiment of the present disclosure. The method 300 initiates with receiving an image signal 50 including one or more images acquired by the camera 30 and, in embodiments, an orientation signal 58 from the orientation sensor 36 of the endoscope 12 (block 302). The method 300 automatically selects a steering target based on the image signal as generally disclosed herein (block 304). In other embodiments, the user may select a target. When the method 300 detects a distal advancement of the endoscope 12 (block 306), the automatic steering is active and the distal end is steered toward the steering target while the endoscope is advancing (block 308). That is, the automatic steering occurs during the distal advancement such that any necessary steering adjustments detected by the system 10 occur during endoscope movement. However, if the method 300 at block 306 detects no motion or no distal advancement of the endoscope 12, the automatic steering is paused (block 312).

Distal movement of the endoscope 12 can be caused by operator pushing of the endoscope 12 from the proximal end, which results in force transferred along the endoscope to the distal end 32. Distal movement can be detected based on the orientation signal 58, the image signal 50, or both. In one example, changes between image frames can be indicative of distal motion. As tracked objects get bigger in the image, the endoscope 12 is getting closer and, thus, moving distally. For cases in which multiple identified features are present in the image signal 50, increasing distance between the multiple tracked center points of the identified features and the center of the image between frames is indicative that the endoscope has moved distally towards the features. In an embodiment, the determination of distal motion, or a lack of distal motion, can be validated based on agreement between the image signal 50 and the orientation signal 58. For example, if both signals indicate distal motion (or absent), then the controller makes the determination that distal movement is present (or absent). If the two signals disagree, then steering may be paused until agreement is achieved in an embodiment. Further, the method 300 may distinguish between distal and proximal motion such that automatic steering is only active during distal advancement in an embodiment and not during endoscope withdrawal (proximal motion). In an embodiment, automatic steering is activated upon a determination that the detected distal movement is above or crosses a certain speed threshold, such that steering is not activated for very small or very slow distal motions. Automatic steering may cause relatively fast changes in the orientation of the distal end, which could cause difficulty in detecting and aligning with very slow distal movement. Thus, activation, or reactivation, of automatic steering can be conditional and based on detection of a minimum speed of distal movement.

By activating automatic steering only during distal motion, the operator has greater control over what parts of the anatomy to view more closely while the endoscope 12 is not advancing. For example, certain region of anatomy may be of interest, and the operator may want to pause distal movement to visually investigate an area. The operator can provide manual inputs to change the orientation of the camera 30 to view the area, such as viewing lesions, polyps, growths, tissue structures, passage walls, e.g., to identify bleeding or structural irregularities. These manual inputs may orient the camera 30 away from the steering target. If automatic steering were active during this manual user investigation, the user input to change the orientation of the camera 30 could conflict with the automatic steering that keeps the distal end 32 aligned with the steering target. Thus, the automatic steering is paused (temporarily deactivated) while the forward motion of the endoscope 12 is also paused, so that the operator does not have to fight the automatic steering to view areas of interest in the anatomy.

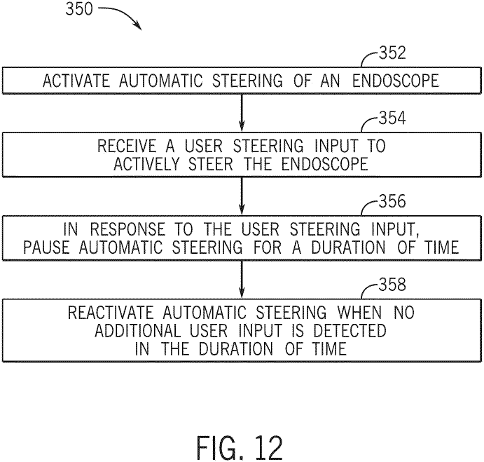

FIG. 12 is a flow diagram of an automatic steering method 350 with a user override that can be used in conjunction with the system 10 and with reference to features discussed in FIGS. 1-4, in accordance with an embodiment of the present disclosure. The method 350 initiates with activation of automatic steering of an endoscope 12 (block 352). The activation can be a default activation, such that powering on the endoscope controller 14 or coupling the endoscope 12 to the endoscope controller 14 activates automatic steering. In embodiments, the activation of automatic steering can be based on user selection of an automatic steering mode via one or more user inputs. In one example, the automatic steering mode can be activated via touching an icon on the display screen 24 or through options in a settings menu.

Once activated, the automatic steering remains active until the controller 14 receives a user steering input to actively steer the endoscope (block 354). The user steering input causes the automatic steering to pause for a duration of time (block 356). Thus, the user steering input to actively steer the endoscope overrides the automatic steering. The automatic steering is reactivated at a subsequent point (block 358), for example after a duration of time during which no additional user steering input is detected. In an embodiment, the automatic steering is fully deactivated (switched into a mode where the automatic steering is not active) rather than paused if the controller receives a large number (above a threshold number) of user steering inputs within a time window. If the user is providing a significant number of steering inputs, the system deactivates automatic steering, and the user can re-activate it later.

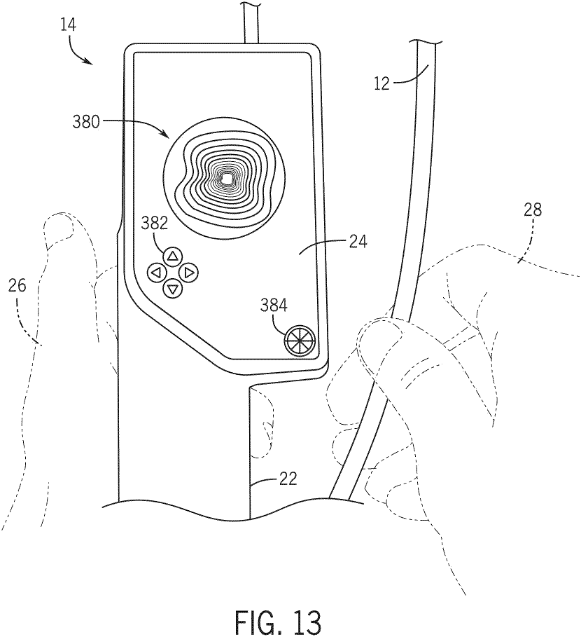

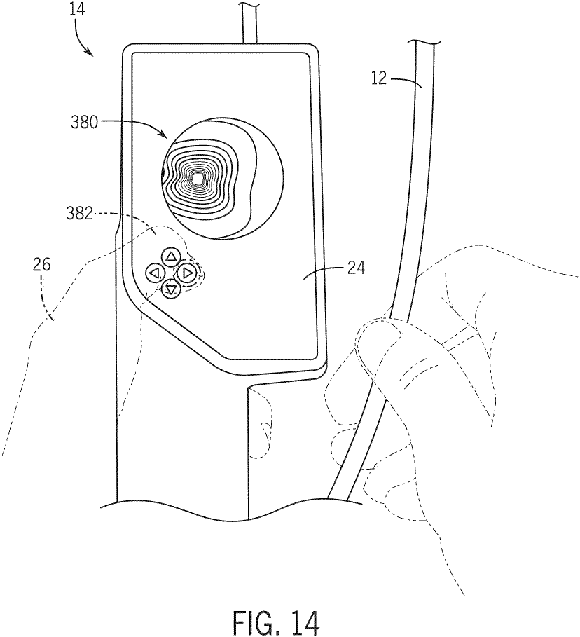

FIGS. 13-14 are schematic illustrations of user interactions to override automatic steering. In the illustrated example of FIG. 13, the laryngoscope operator is holding a wand 22 of the controller 14 in the left hand 26 and manipulating (e.g., advancing) the endoscope 12 with the right hand 28. The display screen 24 shows an endoscope image 380 captured by the endoscope camera. The display screen also shows user steering inputs 382 that the user can interact with on the display screen 24 to change an orientation of the distal end. In the illustrated example, the steering inputs are arrows that control up/down and left/right motion of the distal end. However, other icons and arrangements are possible. For example, the user steering inputs 382 may include a roller ball, virtual joystick, swipe-to-steer (or other touch inputs with or without an associated icon), or other steering input 382. In an embodiment, an automatic steering icon 384, shown as a wheel for purposes of illustration, is active on the display screen. The automatic steering icon 384 indicates whether automatic steering is currently activated (such as by visually distinguishing between active and non-active status, such as by appearing brighter or darker, toggling a strike-out on or off, adjusting colors, or similar changes). The icon 384 is selectable to permit a user to activate or deactivate automatic steering, toggling it on or off.

In FIG. 13, the user’s left hand 26 is not interacting with the user steering inputs 382, and therefore no user steering inputs are received by the controller, and automatic steering is active. In FIG. 14, the user is interacting, via the thumb of the left hand 26, with the user steering inputs 382 to provide a manual input to actively steer the distal end of the endoscope 12. For example, the display screen 24 can include touch sensors that sense the interaction with the user steering inputs 382. In response to user inputs to actively steer, the controller 14 pauses the automatic steering. In the illustrated embodiment, the pausing is indicated by ceasing display of the automatic steering icon 384. However, in other embodiments, the automatic steering icon 383 is retained on the display screen 24 when the automatic steering is paused. User steering operates as an override to the automatic steering to trigger a pause. In an embodiment, the override can be not just in response to sensed steering inputs via the user steering icons 382 but to any sensing of the user interacting with the display screen (such as the user’s thumb or finger touching or being in close proximity to the display screen). However, in other embodiments, the controller 14 can distinguish between user steering inputs (for example where the thumb is touching the steering inputs 382), which can trigger an automatic steering pause, and other non-steering inputs (for example where the thumb is resting or still on the display screen 24), which may not trigger a pause in automatic steering.

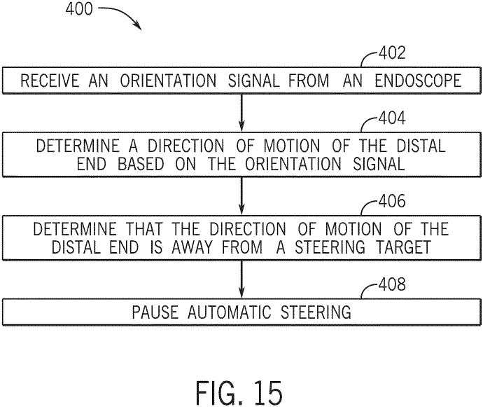

In another example, automatic steering is paused based on contrary detected motion of the endoscope as an indication of manual user steering inputs. Endoscope motions that contradict or are counter to a steering target selected by the steering controller 60 are assumed to be an indication that the user is manually controlling the endoscope to view an area, and this contrary motion can trigger an override or deactivation of the automatic steering. FIG. 15 is a flow diagram of an automatic steering method 400 with an endoscope motion override that can be used in conjunction with the system 10 and with reference to features discussed in FIGS. 1-4, in accordance with an embodiment of the present disclosure. The method 400 initiates by detection of endoscope motion based on receiving an orientation signal 58 from an endoscope (block 402) and determining a direction of motion of the distal tip based on the orientation signal 58 (block 404). When the direction of motion of the distal end is determined to be away from a steering target (block 406), automatic steering is paused or deactivated (block 408). Accordingly, while the steering controller 60 can select a particular steering target, the user can manipulate the endoscope away from the steering target. If the steering controller 60 receives signals indicative of the user fighting the automatic steering, the automatic steering is paused or deactivated.

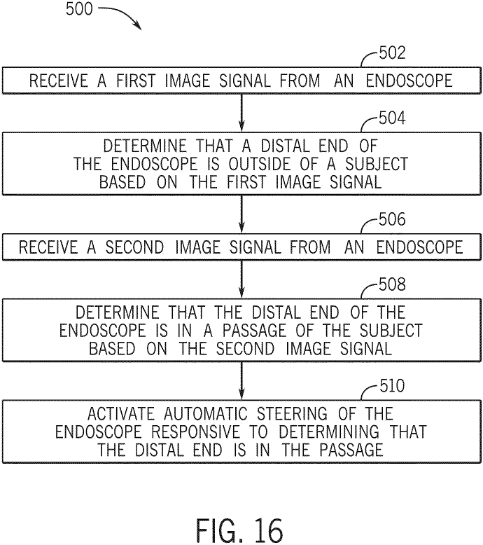

Automatic steering can be paused until the controller detects that the endoscope has entered the patient, to preserve battery life and processing before the clinical procedure has begun. FIG. 16 is a flow diagram of an automatic steering method that automatically activates when the endoscope enters a patient. This method can be used in conjunction with the system 10 and with reference to features discussed in FIGS. 1-4, in accordance with an embodiment of the present disclosure. The method 500 initiates with receiving a first image signal 50 from an endoscope 12 (block 502). The method 500 determines, based on the first image signal 50, that the endoscope 12 is outside of the subject (block 504). In one example, the determination is based on a detected presence of straight (linear) lines in the image. Because straight lines are not typically present in an airway or other interior passage of the patient, identification of one or more straight lines in the image signal 50 indicates that the camera 30 is capturing environmental images and thus the clinical procedure on the patient has not yet begun - which means that automatic steering is not yet needed.

In another example, the determination that the scope is external (viewing the environment, not the patient) can be based on a percentage of red color being below a threshold, because images taken inside the patient are generally redder in color (have a higher percentage of red pixels) than images taken in the external environments (outside the patient). The method 500 receives a second or subsequent image signal 50 from the endoscope 12 (block 506). If the second image signal 50 is determined to be inside of the subject (block 508), automatic steering is activated (block 510). The determination that the endoscope 12 is inside the subject can be based on a percentage of red color being above a threshold, or based on an identification of teeth, a tongue, tonsils, or other anatomical features in the second image signal 50, or based on a user input that the procedure has begun.

In an embodiment, automatic steering can be activated for more curved or challenging portions of the passage, such as the upper airway. For example, the automatic activation can be based on detection of entry into the upper airway, e.g., the endoscope 12 passing through the mouth. After the endoscope 12 has traversed the curved portion of the upper airway and exited through the vocal cords into the relatively straighter trachea, the automatic steering can be deactivated.

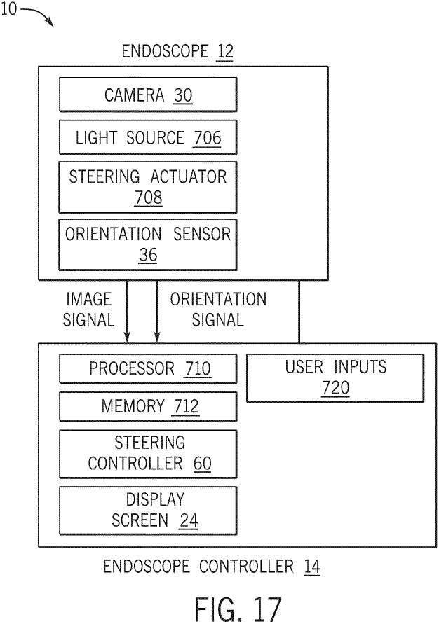

A block diagram of an augmented reality endoscope system 700 is shown in FIG. 17, according to an embodiment. As shown, the system includes the endoscope 12 and the controller 14. The endoscope 12 includes the camera 30, light source 706 (such as an LED shining forward from the distal end of the endoscope), a steering actuator 708 (coupled to one or more distal steerable segments of the endoscope that are steered according to steering instructions), and an orientation sensor 36. The endoscope 12 is connected by a wired (shown) or wireless connection to the endoscope controller 14, which includes a processor 710, hardware memory 712, steering controller 714 (such as a motor or other driver for operating the actuator 708), display screen 24, and one or more user inputs 720, such as touch sensors, switches, or buttons.

In an embodiment, a graphical user interface (GUI) is presented on the display screen 24 of the endoscope controller 14. In an embodiment, the display screen 24 is a touch screen. The GUI receives user inputs by detecting the user’s touch on the screen 24. In an embodiment, the display screen 24 includes a touch screen that is responsive to taps, touches, or proximity gestures from the user. In an embodiment, the user input may additionally or alternatively be provided via user selection from a menu, selection of soft keys, pressing of buttons, operating of a joystick, etc.

In an embodiment, the endoscope 12 includes one, two, or more steerable segments at the distal end of the endoscope. Each articulating segment at the distal end of the endoscope is manipulated by a steering system (such as steering controller 714), which operates an actuator (such as steering actuator 708) according to steering instructions 64.

In an embodiment, the controller 14 together with the endoscope 12 operates as a two-part endoscope, where the controller 14 serves as the handle, display, and user input for the endoscope 12. In an embodiment, the controller 14 is reusable and the endoscope 12 is single-use and disposable, to prevent cross-contamination between patients or caregivers. The controller 14 itself does not need to come into contact with the patient, and it can be wiped and cleaned and ready to use for the next patient, with a new sterile endoscope 12. In an embodiment, the controller 14 is a hand-held wand, and the endoscope 12 is removably connected directly to the wand, for passage of control signals from the wand to the endoscope and video and position signals from the endoscope to the wand. In other embodiments the controller 14 may have other forms or structures, such as a video laryngoscope, table-top display screen, tablet, laptop, puck, or other form factor.

The block diagram of FIG. 17 shows the signal flow between the various devices. In an embodiment, the endoscope 12 sends an image signal (from the camera 30) and an orientation signal (from the orientation sensor 36) to the endoscope controller 14. The endoscope controller 14 receives the image signal and displays image data on the display screen 24.

The orientation sensor 36 is an electronic component that senses the orientation (such as orientation relative to gravity) and/or movement (acceleration) of the distal end of the endoscope. The orientation sensor 36 contains a sensor or a combination of sensors to accomplish this, such as accelerometers, magnetometers, and gyroscopes. The orientation sensor 36 may be an inertial measurement unit (IMU). The orientation sensor 36 detects static orientation and dynamic movement of the distal end of the endoscope and provides the orientation signal 58 indicating a change in the orientation and/or motion of the distal end 32 of the endoscope. The orientation sensor 36 sends this signal to the controller 14. The orientation sensor 36 is located inside the tubular housing of the endoscope 12. As shown in FIG. 1, in an embodiment, the orientation sensor is located very close to the terminus of the distal end of the endoscope, such as behind the camera, to enable the orientation sensor 36 to capture much of the full range of movement of the distal end and camera. In an embodiment, the orientation sensor 36 generates an orientation signal with position coordinates and heading of the distal end of the endoscope 12, and sends the orientation signal to the endoscope controller 14. The data signal from the orientation sensor 36 may be referred to as an orientation signal, movement signal, or position signal.

The feature identification model 54, the steering controller 60, and other functions of the controller 14 can be executed by the processor 710, which may be a chip, a processing chip, a processing board, a chipset, a microprocessor, or similar devices. The processor may include one or more application specific integrated circuits (ASICs), one or more general purpose processors, one or more controllers, FPGA, GPU, TPU, one or more programmable circuits, or any combination thereof. For example, the processor may also include or refer to control circuitry for the display screen. The memory 712 may include volatile memory, such as random access memory (RAM), and/or non-volatile memory, such as read-only memory (ROM). The feature identification model 54 and/or the steering controller 60 may be stored in the memory and accessed by the processor. The memory 712 may include stored instructions, code, logic, and/or algorithms that may be read and executed by the processor to perform the techniques disclosed herein. Certain steps of the flow diagrams discussed herein may be executed by the processor 710 using instructions stored in the memory 712 of the controller 14.

While the present techniques are discussed in the context of endotracheal intubation, it should be understood that the disclosed techniques may also be useful in other types of airway management or clinical procedures. For example, the disclosed techniques may be used in conjunction with placement of other devices within the airway, secretion removal from an airway, arthroscopic surgery, bronchial visualization past the vocal cords (bronchoscopy), tube exchange, lung biopsy, nasal or nasotracheal intubation, etc. In certain embodiments, the disclosed visualization instruments may be used for visualization of anatomy (such as the pharynx, larynx, trachea, bronchial tubes, stomach, esophagus, upper and lower airway, ear-nose-throat, vocal cords), or biopsy of tumors, masses or tissues. The disclosed visualization instruments may also be used for or in conjunction with suctioning, drug delivery, ablation, or other treatments of visualized tissue and may also be used in conjunction with endoscopes, bougies, introducers, scopes, or probes. Further, the disclosed techniques may also be applied to navigation and/or patient visualization using other clinical techniques and/or instruments, such as patient catheterization techniques. By way of example, contemplated techniques include cystoscopy, cardiac catheterization, catheter ablation, catheter drug delivery, or catheter-based minimally invasive surgery.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. An endoscope automatic steering system, comprising:
an endoscope comprising a distal end with a camera producing an image signal; and
an endoscope controller coupled to the endoscope, wherein the endoscope controller:
receives the image signal from the endoscope;
identifies, via a feature identification model, an anatomical feature in the image signal;
selects a steering target based on the identified anatomical feature; and
automatically steers the distal end of the endoscope towards the steering target during distal motion of the distal end of the endoscope.

2. The system of claim 1, wherein the feature identification model identifies the anatomical feature by classifying a subset of pixels in the image signal as the passage and selects a center of the subset of pixels as the steering target.

3. The system of claim 1, wherein the feature identification model identifies the anatomical feature by detecting an object in the image signal and selecting a center of a bounding box around the detected object as the steering target.

4. The system of claim 1, wherein the endoscope controller identifies multiple anatomical features and selects an individual anatomical feature of the identified multiple anatomical features to determine the steering target.

5. The system of claim 1, wherein the endoscope controller:
receives an updated image signal from the endoscope;
identifies the anatomical feature based on the updated image signal;
selects an updated steering target within the anatomical feature;
determines that the distal end is oriented away from the updated steering target; and
generates updated steering instructions to cause the endoscope to automatically steer the distal end towards the updated steering target.

6. The system of claim 1, wherein an indicator representing the steering target is overlaid on a displayed image based on the image signal.

7. The system of claim 1, wherein the steering target is a center or centroid of the identified anatomical feature, wherein the identified anatomical feature comprises a passage.

8. The system of claim 1, wherein the steering target is selected without user steering input.

9. The system of claim 1, wherein the endoscope controller identifies multiple anatomical features and selects an individual anatomical feature comprising a passage from the multiple anatomical features based on user steering input or motion of the endoscope towards the passage.

10. The system of claim 1, wherein the endoscope controller:
determines that the endoscope is inside a subject based on the image signal; and
activates automatic steering to identify the anatomical feature based on the determination.

11. An endoscope automatic steering system, comprising:
an endoscope comprising a steerable distal end with a camera producing an image signal and an orientation sensor producing an orientation signal of an orientation of the steerable distal end;
an endoscope controller that:
receives the image signal and the orientation signal;
automatically selects a steering target of the endoscope based on the image signal;
identifies a distal advancement of the endoscope based on the orientation signal or the image signal or both; and
generates instructions to automatically steer the distal end of the endoscope towards the steering target during the distal advancement.

12. The system of claim 11, wherein the endoscope controller:
determines, based on the orientation signal or the image signal or both, that the distal advancement has stopped; and
pauses automatically steering the distal end while the distal advancement remains stopped.

13. The system of claim 12, wherein the endoscope controller receives one or more user steering inputs while the distal advancement has stopped and changes an orientation of the distal end based on the one or more user steering inputs.

14. The system of claim 11, wherein the endoscope controller automatically steers the distal end only when an automatic steering mode is activated.

15. The system of claim 11, wherein the endoscope controller automatically selects the steering target by identifying features of the image signal characteristic of a passage and selecting a center of the passage as the steering target.

16. The system of claim 11, comprising an automatic steering icon displayed on a display screen.

17. An endoscope automatic steering method, comprising:
automatically steering an endoscope towards a steering target in a passage of a subject;
receiving a user steering input to actively steer the endoscope;
pausing automatically steering of the endoscope based on the user steering input; and
resuming automatically steering the endoscope towards the steering target after a predetermined time period has passed during which no additional user steering inputs are received.

18. The method of claim 17, wherein the user steering input is received via a touch screen of an endoscope controller.

19. The method of claim 17, comprising determining that the endoscope is inside the passage of the subject based on an image signal from the endoscope and activating automatically steering the endoscope based on the determining.

20. The method of claim 19, wherein determining that the endoscope is inside the patient comprises determining that the image signal is above a threshold of red percentage or that the image signal does not have straight lines.
